(19) United States
(12) Patent Application Publication
(10) Pub. No.: US 2025/0288731 A1
(43) Pub. Date: Sep. 18, 2025

(54) COATING COMPOSITION FOR PROTECTION OF ENDOSCOPE

(71) Applicant: Lynk Solutec Inc, Seoul (KR)

(72) Inventors: Jungmok SEO, Seoul (KR); Yeontaek LEE, Seoul (KR)

(73) Assignee: Lynk Solutec Inc, Seoul (KR)

(21) Appl. No.: 19/066,705

(22) Filed: Feb. 28, 2025

(30) Foreign Application Priority Data
Mar. 13, 2024 (KR) 10-2024-0035243

(51) Int. Cl. A61L 29/14; A61B 1/00; A61L 29/08

(52) U.S. Cl. CPC A61L 29/14; A61B 1/0011; A61B 1/00137; A61L 29/085; A61L 2400/10; A61L 2420/06

(57) ABSTRACT

Provided is a coating composition for visibility of an endoscope, and more specifically, the coating composition includes a stock solution and a diluted solution, and the stock solution includes an aliphatic hydrocarbon solvent; a siloxane compound; an alcohol; one or more amino silanes; and a silane-based oligomer.

### CROSS-REFERENCE TO RELATED APPLICATIONS

This application claims the priority of Korean Patent Application No. 10-2024-0035243 filed on Mar. 13, 2024, in the Korean Intellectual Property Office, the disclosure of which is incorporated herein by reference.

### BACKGROUND

### Field

The present disclosure relates to a coating composition for visibility of an endoscope, and more particularly, to a coating composition including a stock solution and a diluted solution, in which the stock solution includes an aliphatic hydrocarbon solvent; a siloxane compound; an alcohol; one or more amino silanes; and a silane-based oligomer.

### Description of the Related Art

An endoscope is a medical device generally used with an endoscope camera, and is used in various medical fields to visually observe and diagnose the inside of a patient during or before and after surgery to perform surgery.

In the endoscope, “visibility” is a very important factor. When the visibility of the endoscope is not sufficiently secured, it may be difficult to conduct a diagnosis or surgery smoothly, and the success of the surgery may be hindered, so that continuous research and improvement are required for better visibility of the endoscope.

Currently, an endoscope cap has been used as a method for improving the visibility of the endoscope. When the endoscope cap is mounted on the top of the endoscope, the endoscope cap serves to enable the visibility because a distance between a tip of the endoscope equipped with a camera and the mucosa, internal organs, etc. of a patient is increased. However, the endoscope cap has a function of securing the visibility that would not be seen because the mucosa was in contact if the cap had not been mounted by decreasing the distance between the tip of the endoscope and the mucosa, but there is still a problem of the visibility blocked due to fogging and bleeding during the endoscope. To solve this problem, a doctor needs to directly wash a lens or apply an anti-fogging agent at short intervals, which increases the surgery time by about 20%, increases the fatigue of the medical staff, and has the risk of infection and inflammation in the patient.

Accordingly, the present inventors developed a special coating composition that fundamentally did not adhere to moisture, mucus, and bacteria including blood while conducting research to solve the above problems, discovered that a coating layer was formed on an endoscope cap and/or an endoscope tip using the coating composition to have an excellent visibility effect of the endoscope, and then completed the present disclosure.

In this regard, Korean Patent Registration No. 10-2407163 discloses an endoscope cap with anti-bioadhesion coating.

### SUMMARY

The present disclosure has been made to solve the problems of the related art, and an object of the present disclosure is to provide a coating composition for visibility of an endoscope including a stock solution and a diluted solution.

Another object of the present disclosure is to provide a film for visibility of an endoscope including a base film; and a coating composition coated on at least one surface of the base film, in which the coating composition includes a stock solution and a diluted solution, and the stock solution includes an aliphatic hydrocarbon solvent; a siloxane compound; an alcohol; one or more amino silanes; and a silane-based oligomer.

As a technical solution for achieving the above-mentioned technical problem, one aspect of the present disclosure provides a coating composition for visibility of an endoscope, including a stock solution and a diluted solution, in which the stock solution includes an aliphatic hydrocarbon solvent; a siloxane compound; an alcohol; one or more amino silanes; and a silane-based oligomer.

The diluted solution may include an organosilicon compound.

The weight mixing ratio of the stock solution and the diluted solution may be 1:45 to 55.

The coating composition may be coated on a distal part of the endoscope.

Another aspect of the present disclosure provides a film for visibility of an endoscope including a base film; and a coating composition coated on at least one surface of the base film, in which the coating composition includes a stock solution and a diluted solution, and the stock solution includes an aliphatic hydrocarbon solvent; a siloxane compound; an alcohol; one or more amino silanes; and a silane-based oligomer.

The film for visibility of the endoscope may further include a silane-based lubricant coated on at least one surface of the base film.

According to the present disclosure, the coating composition may have an effect of preventing fogging and adhesion of biological materials such as blood when applied to an endoscope.

In addition, the coating composition may have a function of protecting the endoscope by being applied to the surface of the distal part surfaces of components like the lens, cap, etc. of the endoscope in various forms such as a coating, a film, and the like.

The effects of the present disclosure are not limited to the aforementioned effects, and other effects, which are not mentioned above, will be apparently understood to a person having ordinary skill in the art from the following description.

The objects to be achieved by the present disclosure, the means for achieving the objects, and the effects of the present disclosure described above do not specify essential features of the claims, and, thus, the scope of the claims is not limited to the disclosure of the present disclosure.

### BRIEF DESCRIPTION OF THE DRAWINGS

The above and other aspects, features and other advantages of the present disclosure will be more clearly understood from the following detailed description taken in conjunction with the accompanying drawings, in which:

FIG. 1 is a photograph showing an XPS survey spectrum of a coating composition according to Example of the present disclosure;

FIG. 2 is a graph showing light transmittance of a glass plate coated with a coating composition according to Example of the present disclosure;

FIGS. 3A and 3B are photographs showing results of a blood adhesion prevention experiment of coating compositions according to Example and Comparative Example of the present disclosure, respectively;

FIG. 4 is a photograph showing results of a marine organism adhesion prevention experiment of a coating composition according to Example of the present disclosure;

FIG. 5 is a photograph showing endoscope visibility performance of a coating composition according to Example of the present disclosure; and

FIG. 6 is a photograph showing visibility performance of a general endoscope according to Comparative Example of the present disclosure.

### DETAILED DESCRIPTION OF THE EMBODIMENT

Hereinafter, the present disclosure will be described in more detail. However, the present disclosure may be embodied in various different forms, and the present disclosure is not limited by embodiments described herein, and the present disclosure will be only defined by claims to be described below.

Terms used in the present disclosure are used only to describe specific embodiments, and are not intended to limit the present disclosure. A singular expression includes a plural expression unless otherwise defined differently in a context.

Throughout the present specification, unless explicitly described to the contrary, ‘comprising’ a certain component means further comprising another component other than excluding the other component.

A first aspect of the present disclosure provides a coating composition for visibility of an endoscope, including a stock solution and a diluted solution, in which the stock solution includes an aliphatic hydrocarbon solvent; a siloxane compound; an alcohol; one or more amino silanes; and a silane-based oligomer.

Hereinafter, the coating composition for visibility of the endoscope according to the first aspect of the present disclosure will be described in detail.

In one embodiment of the present disclosure, the coating composition may include a stock solution and a diluted solution, and at this time, the stock solution may include an aliphatic hydrocarbon solvent, a siloxane compound, an alcohol, one or more amino silanes, and a silane-based oligomer.

In one embodiment of the present disclosure, the aliphatic hydrocarbon solvent may include a C₇ to C₁₂ aliphatic hydrocarbon solvent, and preferably a C₉ to C₁₂ aliphatic hydrocarbon solvent. In addition, the aliphatic hydrocarbon solvent may have a boiling range of about 140° C. to 220° C.

In one embodiment of the present disclosure, the content of the aliphatic hydrocarbon solvent may be 20 to 50 parts by weight, preferably 24 to 25 parts by weight, and more preferably 30 to 40 parts by weight, based on 100 parts by weight of the stock solution.

In one embodiment of the present disclosure, the siloxane compound may include a compound selected from the group consisting of polydimethylsiloxane, polyvinylsiloxane, polyphenylmethylsiloxane, hexamethyldisiloxane, and combinations thereof, and preferably may include a compound selected from the group consisting of methyl-terminated polydimethylsiloxane, vinyl-terminated polydimethylsiloxane, hydroxy-terminated polydimethylsiloxane, carboxy-terminated polydimethylsiloxane, and combinations thereof.

In one embodiment of the present disclosure, the content of the siloxane compound may be 30 to 60 parts by weight, preferably 35 to 55 parts by weight, and more preferably 40 to 50 parts by weight, based on 100 parts by weight of the stock solution.

In one embodiment of the present disclosure, the alcohol may be selected from the group consisting of, for example, methanol, ethanol, isopropanol, cyclohexanol, benzyl alcohol, 2-octanol, ethylene glycol, propylene glycol, glycerol, and combinations thereof.

In one embodiment of the present disclosure, the content of the alcohol may be 5 to 25 parts by weight, preferably 7 to 13 parts by weight, and more preferably 10 to 20 parts by weight, based on 100 parts by weight of the stock solution.

In one embodiment of the present disclosure, the amino silane may include amino silane including at least one selected from the group consisting of bis[(3-trimethoxysilyl)propyl]amine silane, N-beta-(aminoethyl)-gamma-aminopropyl-trimethoxy silane, N-(3-(trimethoxysilyl)propyl)-1,2-ethanediamine silane, N,N′-bis(3-(trimethoxysilyl)propyl)-1,2-ethanediamine silane, and N—N-bis(3-(trimethylsiloxy)propyl)-1,2-ethanediamine silane.

In one embodiment of the present disclosure, the content of the one or more amino silanes may be 5 to 20 parts by weight, preferably 7 to 17 parts by weight, and more preferably 10 to 15 parts by weight, based on 100 parts by weight of the stock solution.

In one embodiment of the present disclosure, the silane-based oligomer may include a compound formed from monomers selected from the group consisting of dimethyldimethoxysilane, vinylmethyldimethoxysilane, n-octylmethyldimethoxysilane, n-octadecylmethyldimethoxysilane, methyldimethoxysilane, 3-chloropropylmethyldimethoxysilane, 2-chloroethylmethyldimethoxysilane, allyldimethoxysilane, (3,3,3-trifluoropropyl)methyldimethoxysilane, n-propylmethyldimethoxysilane, chloromethylmethyldimethoxysilane, di-n-octyldimethoxysilane, vinyl(chloromethyl)dimethoxysilane, methylcyclohexyldiethoxysilane, vinylmethyldiethoxysilane, 1-(triethoxysilyl)-2-(diethoxymethylsilyl)ethane, n-octylmethyldiethoxysilane, octaethoxy-1,3,5-trisilapentane, n-octadecylmethyldiethoxysilane, methacryloxypropylmethyldiethoxysilane, 2-hydroxy-4-(3-methyldiethoxysilylpropoxy)diphenylketone, (3-glycidoxypropyl)methyldiethoxysilane, dodecylmethyldiethoxysilane, dimethyldiethoxysilane, diethyldiethoxysilane, 1,1-diethoxy-1-silacyclopent-3-ene, chloromethylmethyldiethoxysilane, bis(methyldiethoxysilylpropyl)amine, 3-aminopropylmethyldiethoxysilane, (methacryloxymethyl)methyldiethoxysilane, 1,2-bis(methyldiethoxysilyl)ethane, and diisobutyldiethoxysilane, vinyltrimethoxysilane, vinyltriethoxysilane, benzyltriethoxysilane, butenyltriethoxysilane, (triethoxysilyl)cyclohexane, 0-(vinyloxybutyl)-N-triethoxysilylpropylcarbamate, 10-undecenyltrimethoxysilane, n-(3-trimethoxysilylpropyl)pyrrole, N-[5-(trimethoxysilyl)-2-aza-1-oxopentyl]caprolactam, (3,3,3-trifluoropropyl)trimethoxysilane, triethoxysilylundecanal ethylene glycol acetal, (S)—N-triethoxysilylpropyl-O-menthocarbamate, triethoxysilylpropylethylcarbamate, N-(3-triethoxysilylpropyl)-4,5-dihydroimidazole, (3-triethoxysilylpropyl)-t-butylcarbamate, styrylethyltrimethoxysilane, 2-(4-pyridylethyl) triethoxysilane, n-propyltrimethoxysilane, n-propyltriethoxysilane, (S)—N-1-phenylethylN′-triethoxysilylpropylurea, (R)—N-1-phenylethyl-N′-triethoxysilylpropylurea, N-phenylaminopropyltrimethoxysilane, N-phenylaminomethyltriethoxysilane, phenethyltrimethoxysilane, pentyltriethoxysilane, n-octyltrimethoxysilane, n-octyltriethoxysilane, 7-octenyltrimethoxysilane, S-(octanoyl)mercaptopropyltriethoxysilane, n-octadecyltrimethoxysilane, n-octadecyltriethoxysilane, methyltrimethoxysilane, methyltriethoxysilane, N-methylaminopropyltrimethoxysilane, 3-methoxypropyltrimethoxysilane, methacryloxypropyltrimethoxysilane, methacryloxypropyltriethoxysilane, methacryloxymethyltrimethoxysilane, methacryloxymethyltriethoxysilane, and O-(methacryloxyethyl)-N-(triethoxysilylpropyl)carbamate, tetramethoxysilane and/or tetraethoxysilane, octamethylcyclotetrasiloxane, and combinations thereof.

In one embodiment of the present disclosure, the content of the silane-based oligomer may be 0.1 to 1.0 parts by weight based on 100 parts by weight of the stock solution.

In one embodiment of the present disclosure, the diluted solution may be an organosilicon compound, and the organosilicon compound may include a compound selected from the group consisting of hexamethyldisiloxane, 1,1,3,3-tetramethyldisiloxane, vinyltrimethylsilane, methyltrimethylsilane, hexamethyldisilane, methylsilane, dimethylsilane, trimethylsilane, diethylsilane, propylsilane, phenylsilane, vinyltriethoxysilane, vinyltrimethoxysilane, tetramethoxysilane, tetraethoxysi lane, phenyltrimethoxysilane, methyltriethoxysilane, octamethylcyclotetrasiloxane, and combinations thereof.

In one embodiment of the present disclosure, the weight mixing ratio of the stock solution and the diluted solution may be 1:45 to 55, specifically 1:47 to 52, and more specifically 1:48 to 51. At this time, if the weight mixing ratio of the stock solution and the diluted solution is less than 1:45, the coating property of the coating composition may be deteriorated, and the transmittance may be reduced, and thus when the distal part of the endoscope is coated with the coating composition, there may be a problem that the performance of the endoscope lens may be reduced. In addition, if the weight mixing ratio of the stock solution and the diluted solution is more than 1:55, the content of the stock solution to be contained may be too small, so that a sufficient protection function, a blood adhesion prevention effect, or a visibility function of the endoscope may not be sufficiently exhibited.

In one embodiment of the present disclosure, the coating composition may further include a silane-based lubricant. At this time, in one embodiment of the present disclosure, in the coating composition, the silane-based lubricant may be mixed with the stock solution and the diluted solution and coated together, or the coating composition in which the stock solution and the diluted solution are mixed may be first coated to form an intermediate coating layer, and then an oil film layer may also be formed on the coating layer of the stock solution and the diluted solution using the silane-based lubricant, and preferably, the oil film layer may be coated on the coating layer of the stock solution and the diluted solution using the silane-based lubricant.

In one embodiment of the present disclosure, the silane-based lubricant may include a lubricant selected from the group consisting of a polydimethylsiloxane-based lubricant, a trimethylsilyl-terminated polydimethylsiloxane-based lubricant, a vinyldimethylsilyl-terminated polydimethylsiloxane-based lubricant, an epoxycyclohexyldimethylsilyl-terminated polydimethylsiloxane-based lubricant, a glycidoxypropyldimethylsilyl-terminated polydimethylsiloxane-based lubricant, a p-styryldimethylsilyl-terminated polydimethylsiloxane-based lubricant, a methacryloxypropyldimethylsilyl-terminated polydimethylsiloxane-based lubricant, an acryloxydimethylsilyl-terminated polydimethylsiloxane-based lubricant, an aminopropyldimethylsilyl-terminated polydimethylsiloxane-based lubricant, a 3-isocyanatopropyldimethylsilyl-terminated polydimethylsiloxane-based lubricant, a 3-mercaptopropyldimethylsilyl-terminated polydimethylsiloxane-based lubricant, a 3-ureidopropyldimethylsilyl-terminated polydimethylsiloxane-based lubricant, an ethyldimethylsilyl-terminated polydimethylsiloxane-based lubricant, a propyldimethylsilyl-terminated polydimethylsiloxane-based lubricant, a hexyldimethylsilyl-terminated polydimethylsiloxane-based lubricant, an octyldimethylsilyl-terminated polydimethylsiloxane-based lubricant, a decyldimethylsilyl-terminated polydimethylsiloxane-based lubricant, a phenyldimethylsilyl-terminated polydimethylsiloxane-based lubricant, and combinations thereof.

In one embodiment of the present disclosure, the coating composition may be coated on the distal part of the endoscope. At this time, the distal part refers to a portion disposed(located) at the tip of the endoscope, and for example, the distal part of the endoscope may refer to lens, an endoscope cap, etc., but is not limited thereto, and may refer to a distal part of the endoscope that requires visibility of the endoscope by preventing fogging and adhesion of biological materials such as blood, or a distal part of the endoscope that requires protection from damage to the endoscope by colliding with other surgical tools during surgery.

In one embodiment of the present disclosure, the coating composition may be applied to the distal part of the endoscope in various forms depending on a purpose of use, a type of endoscope, a coating method, etc. Specifically, in one embodiment of the present disclosure, the coating composition may be directly coated on an endoscope cap, may be coated on a base film to adhere to an endoscope lens in the form of a film, and the coating composition may be injection-molded into the form of an endoscope cap to be used in a form of covering the endoscope or injection-molded into the form of a distal part of the endoscope to be used in a form of covering a lens and a thin body portion of the endoscope, and may also be directly coated on the distal part of the endoscope itself, but is not limited thereto.

Meanwhile, the coating composition has high transmittance and has also effects of preventing biological materials such as blood and fogging, thereby providing an endoscope visibility effect, and has excellent physical properties, thereby providing a function of protecting the distal part of the endoscope.

A second aspect of the present disclosure provides a film for visibility of an endoscope including a base film; and a coating composition laminated on at least one surface of the base film and including a stock solution and a diluted solution, in which the stock solution includes an aliphatic hydrocarbon solvent; a siloxane compound; an alcohol; one or more amino silanes; and a silane-based oligomer.

Detailed descriptions of portions overlapping with those of the first aspect of the present disclosure have been omitted, but the contents described for the first aspect of the present disclosure can be equally applied even if the description thereof has been omitted from the second aspect.

Hereinafter, the film for visibility of the endoscope according to the second aspect of the present disclosure will be described in detail.

In one embodiment of the present disclosure, the base film is not particularly limited in its type, but preferably, may be used with a plastic film having excellent transmittance and transparency. For example, the base film may be a base film made of polyethylene, polypropylene, polystyrene, polyvinyl chloride, polychlorinated biphenyl, polyethylene terephthalate, polyvinyl alcohol, polycarbonate, polybutylene terephthalate, polyethylene naphthalate, polyamide, polyacetal, polyphenylene ether, polyamideimide, polyetherimide, polyetheretherketone, polyimide, polytetrafluoroethylene, liquid crystal polymer, fluororesin, or a copolymer or mixture thereof.

In one embodiment of the present disclosure, the aliphatic hydrocarbon solvent may include a C₇ to C₁₂ aliphatic hydrocarbon solvent, and preferably a C₉ to C₁₂ aliphatic hydrocarbon solvent. In addition, the aliphatic hydrocarbon solvent may have a boiling range of about 140° C. to 220° C.

In one embodiment of the present disclosure, the content of the aliphatic hydrocarbon solvent may be 20 to 50 parts by weight, preferably 24 to 25 parts by weight, and more preferably 30 to 40 parts by weight, based on 100 parts by weight of the stock solution.

In one embodiment of the present disclosure, the siloxane compound may include a compound selected from the group consisting of polydimethylsiloxane, polyvinylsiloxane, polyphenylmethylsiloxane, hexamethyldisiloxane, and combinations thereof, and preferably may include a compound selected from the group consisting of methyl-terminated polydimethylsiloxane, vinyl-terminated polydimethylsiloxane, hydroxy-terminated polydimethylsiloxane, carboxy-terminated polydimethylsiloxane, and combinations thereof.

In one embodiment of the present disclosure, the content of the siloxane compound may be 30 to 60 parts by weight, preferably 35 to 55 parts by weight, and more preferably 40 to 50 parts by weight, based on 100 parts by weight of the stock solution.

In one embodiment of the present disclosure, the alcohol may be selected from the group consisting of, for example, methanol, ethanol, isopropanol, cyclohexanol, benzyl alcohol, 2-octanol, ethylene glycol, propylene glycol, glycerol, and combinations thereof.

In one embodiment of the present disclosure, the content of the alcohol may be 5 to 25 parts by weight, preferably 7 to 13 parts by weight, and more preferably 10 to 20 parts by weight, based on 100 parts by weight of the stock solution.

In one embodiment of the present disclosure, the amino silane may include amino silane including at least one selected from the group consisting of bis[(3-trimethoxysilyl)propyl]amine silane, N-beta-(aminoethyl)-gamma-aminopropyl-trimethoxy silane, N-(3-(trimethoxysilyl)propyl)-1,2-ethanediamine silane, N,N′-bis(3-(trimethoxysilyl)propyl)-1,2-ethanediamine silane, and N—N-bis(3-(trimethylsiloxy)propyl)-1,2-ethanediamine silane.

In one embodiment of the present disclosure, the content of the one or more amino silanes may be 5 to 20 parts by weight, preferably 7 to 17 parts by weight, and more preferably 10 to 15 parts by weight, based on 100 parts by weight of the stock solution.

In one embodiment of the present disclosure, the silane-based oligomer may include a compound formed from monomers selected from the group consisting of dimethyldimethoxysilane, vinylmethyldimethoxysilane, n-octylmethyldimethoxysilane, n-octadecylmethyldimethoxysilane, methyldimethoxysilane, 3-chloropropylmethyldimethoxysilane, 2-chloroethylmethyldimethoxysilane, allyldimethoxysilane, (3,3,3-trifluoropropyl)methyldimethoxysilane, n-propylmethyldimethoxysilane, chloromethylmethyldimethoxysilane, di-n-octyldimethoxysilane, vinyl(chloromethyl)dimethoxysilane, methylcyclohexyldiethoxysilane, vinylmethyldiethoxysilane, 1-(triethoxysilyl)-2-(diethoxymethylsilyl)ethane, n-octylmethyldiethoxysilane, octaethoxy-1,3,5-trisilapentane, n-octadecylmethyldiethoxysilane, methacryloxypropylmethyldiethoxysilane, 2-hydroxy-4-(3-methyldiethoxysilylpropoxy)diphenylketone, (3-glycidoxypropyl)methyldiethoxysilane, dodecylmethyldiethoxysilane, dimethyldiethoxysilane, diethyldiethoxysilane, 1,1-diethoxy-1-silacyclopent-3-ene, chloromethylmethyldiethoxysilane, bis(methyldiethoxysilylpropyl)amine, 3-aminopropylmethyldiethoxysilane, (methacryloxymethyl)methyldiethoxysilane, 1,2-bis(methyldiethoxysilyl)ethane, and diisobutyldiethoxysilane, vinyltrimethoxysilane, vinyltriethoxysilane, benzyltriethoxysilane, Butenyltriethoxysilane, (triethoxysilyl)cyclohexane, 0-(vinyloxybutyl)-N-triethoxysilylpropylcarbamate, 10-undecenyltrimethoxysilane, n-(3-trimethoxysilylpropyl)pyrrole, N-[5-(trimethoxysilyl)-2-aza-1-oxopentyl]caprolactam, (3,3,3-trifluoropropyl)trimethoxysilane, triethoxysilylundecanal ethylene glycol acetal, (S)—N-triethoxysilylpropyl-O-menthocarbamate, triethoxysilylpropylethylcarbamate, N-(3-triethoxysilylpropyl)-4,5-dihydroimidazole, (3-triethoxysilylpropyl)-t-butylcarbamate, styrylethyltrimethoxysilane, 2-(4-pyridylethyl)triethoxysilane, n-propyltrimethoxysilane, n-propyltriethoxysilane, (S)—N-1-phenylethylN′-triethoxysilylpropylurea, (R)—N-1-phenylethyl-N′-triethoxysilylpropylurea, N-phenylaminopropyltrimethoxysilane, N-phenylaminomethyltriethoxysilane, phenethyltrimethoxysilane, pentyltriethoxysilane, n-octyltrimethoxysilane, n-octyltriethoxysilane, 7-octenyltrimethoxysilane, S-(octanoyl)mercaptopropyltriethoxysilane, n-octadecyltrimethoxysilane, n-octadecyltriethoxysilane, methyltrimethoxysilane, methyltriethoxysilane, N-methylaminopropyltrimethoxysilane, 3-methoxypropyltrimethoxysilane, methacryloxypropyltrimethoxysilane, methacryloxypropyltriethoxysilane, methacryloxymethyltrimethoxysilane, methacryloxymethyltriethoxysilane, and O-(methacryloxyethyl)-N-(triethoxysilylpropyl)carbamate, tetramethoxysilane and/or tetraethoxysilane, octamethylcyclotetrasiloxane and combinations thereof.

In one embodiment of the present disclosure, the content of the silane-based oligomer may be 0.1 to 1.0 parts by weight based on 100 parts by weight of the stock solution.

In one embodiment of the present disclosure, the diluted solution may be an organosilicon compound, and the organosilicon compound may include a compound selected from the group consisting of hexamethyldisiloxane, 1,1,3,3-tetramethyldisiloxane, vinyltrimethylsilane, methyltrimethylsilane, hexamethyldisilane, methylsilane, dimethylsilane, trimethylsilane, diethylsilane, propylsilane, phenylsilane, vinyltriethoxysilane, vinyltrimethoxysilane, tetramethoxysilane, tetraethoxysi lane, phenyltrimethoxysilane, methyltriethoxysilane, octamethylcyclotetrasiloxane, and combinations thereof.

In one embodiment of the present disclosure, the weight mixing ratio of the stock solution and the diluted solution may be 1:45 to 55, specifically 1:47 to 52, and more specifically 1:48 to 51. At this time, if the weight mixing ratio of the stock solution and the diluted solution is less than 1:45, there may be a problem that the coating property of the coating composition may be deteriorated and the transmittance may be reduced, and if the weight mixing ratio of the stock solution and the diluted solution is more than 1:55, the content of the stock solution included may be too small, and thus a sufficient protective function, a blood adhesion prevention effect, or a visibility function of the endoscope may not be sufficiently exhibited.

In one embodiment of the present disclosure, the film for visibility of the endoscope may further include a silane-based lubricant applied to at least one surface of the base film. Preferably, the silane-based lubricant may be further applied to one surface of the base film on which the coating composition is coated, and more preferably, the coating composition is coated on the base film, and then applied to the upper portion of the coating layer to form an oil film layer.

In one embodiment of the present disclosure, the silane-based lubricant may include a lubricant selected from the group consisting of a polydimethylsiloxane-based lubricant, a trimethylsilyl-terminated polydimethylsiloxane-based lubricant, a vinyldimethylsilyl-terminated polydimethylsiloxane-based lubricant, an epoxycyclohexyldimethylsilyl-terminated polydimethylsiloxane-based lubricant, a glycidoxypropyldimethylsilyl-terminated polydimethylsiloxane-based lubricant, a p-styryldimethylsilyl-terminated polydimethylsiloxane-based lubricant, a methacryloxypropyldimethylsilyl-terminated polydimethylsiloxane-based lubricant, an acryloxydimethylsilyl-terminated polydimethylsiloxane-based lubricant, an aminopropyldimethylsilyl-terminated polydimethylsiloxane-based lubricant, a 3-isocyanatopropyldimethylsilyl-terminated polydimethylsiloxane-based lubricant, a 3-mercaptopropyldimethylsilyl-terminated polydimethylsiloxane-based lubricant, a 3-ureidopropyldimethylsilyl-terminated polydimethylsiloxane-based lubricant, an ethyldimethylsilyl-terminated polydimethylsiloxane-based lubricant, a propyldimethylsilyl-terminated polydimethylsiloxane-based lubricant, a hexyldimethylsilyl-terminated polydimethylsiloxane-based lubricant, an octyldimethylsilyl-terminated polydimethylsiloxane-based lubricant, a decyldimethylsilyl-terminated polydimethylsiloxane-based lubricant, a phenyldimethylsilyl-terminated polydimethylsiloxane-based lubricant, and combinations thereof.

In one embodiment of the present disclosure, the method of applying the coating composition to the base film includes roll coating, spin coating, dip coating, flow coating, spray coating, etc., but is not limited thereto.

In one embodiment of the present disclosure, the film for visibility of the endoscope may be further cured by a method such as ultraviolet irradiation, plasma treatment, or heat treatment after coating the coating composition on the base film.

In one embodiment of the present disclosure, the film coated with the coating composition may adhere to an endoscope lens. At this time, the adhered film may protect the endoscope lens or the distal part of the endoscope to be prevented from being damaged.

Hereinafter, Examples of the present disclosure will be described in detail to be easily implemented by those skilled in the art. However, the present disclosure can be implemented in various different forms and is not limited to Examples described herein.

### Example 1. Coating of Glass Plate Using Coating Composition

### 1. Preparation of Coating Composition

According to ingredients and contents shown in Table 1 below, a stock solution of a coating composition for visibility of an endoscope was prepared, and the prepared stock solution was mixed with hexamethyldisiloxane as a diluted solution at a weight mixing ratio of 1:49 to prepare a coating composition.

TABLE 1

| | | Content |
|---|---|---|
| NO | Ingredient | (wt %) |
| 1 | Medium aliphatic solvent naphtha (petroleum) | 35 |
| 2 | Polydimethylsiloxane hydroxy-terminated | 43.5 |
| 3 | Isopropanol | 15 |
| 4 | N-(3-(Trimethoxysilyl) propyl)-1,2-ethanediamine | 5 |
| 5 | N,N′-bis(3-(trimethoxysilyl) propyl)-1,2-ethanediamine | 0.5 |
| 6 | N,N-Bis(3-(Trimethylsiloxy) propyl)-1,2-ethanediamine | 0.5 |
| 7 | Oligomers of (ethylenediaminepropyl) trimethoxysilane | 0.5 |

### 2. Coating of Glass Plate

A glass plate was prepared as a coating base, and the glass plate was immersed in the coating composition prepared in 1. above, taken out, and dried for 5 to 10 seconds, and then coated with the coating composition of 1. above, and coated again with polydimethyl siloxanes and silicones as a lubricant in the same manner to coat an oil film layer.

### Example 2. Coating of Endoscope Cap Using Coating Composition

An endoscope cap was immersed in the coating composition prepared in 1. of Example 1, taken out, and dried for 5 to 10 seconds, and then coated with the coating composition of 1. of Example 1, and coated again with polydimethyl siloxanes and silicones as a lubricant in the same manner to coat an oil film layer.

### Comparative Example 1. Preparation of General Glass Plate

An uncoated glass plate, which was the base used in 2. of Example 1, was prepared.

### Comparative Example 2. Preparation of General Endoscope Cap

An uncoated endoscope cap used in Example 2 was prepared.

### Experimental Example 1. Analysis of X-Ray Photoelectron Spectroscopy (XPS) of Coating Composition

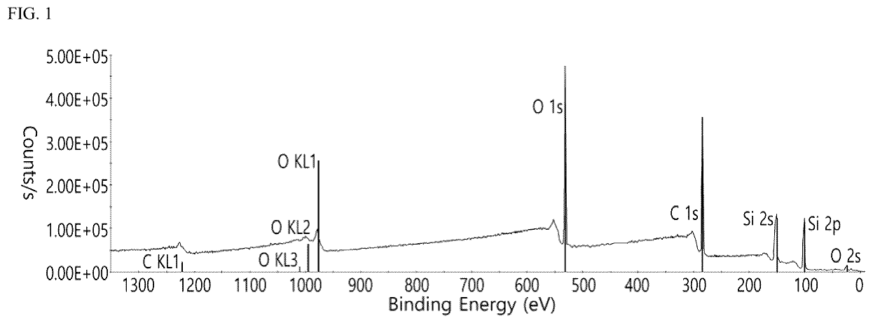

X-ray photoelectron spectroscopy (XPS) analysis was performed on the coating composition prepared in Example 1, and the results were shown in FIG. 1.

As a result of the analysis, it was confirmed that peaks appeared in C KL1, O KL3, O KL2, O KL1, O is, C 1 s, Si 2 s, Si 2p, and O 2 s.

Through this, it was confirmed that the ingredients according to Table 1 of Example 1 were included.

### Experimental Example 2. Analysis of Light Transmittance of Coating Composition

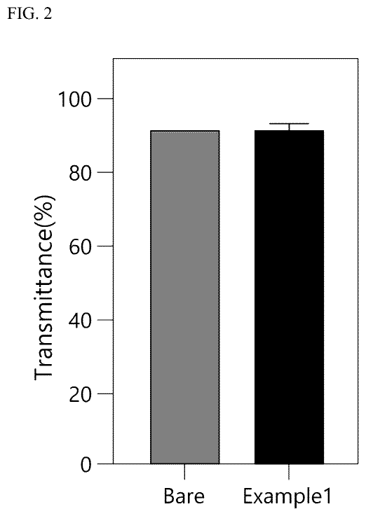

The light transmittance of the glass plate coated with the coating composition of Example 1 was measured, and the results were shown in FIG. 2. At this time, a graph shown in FIG. 2 represented the average value at UV-Vis 400 nm to 800 nm.

As shown in FIG. 2, it was confirmed that the coating composition according to Example 1 had excellent transmittance (transparency), and thus was suitable for use in endoscope lenses, endoscope caps, etc.

### Experimental Example 3. Confirmation of Blood Adhesion Prevention Effect

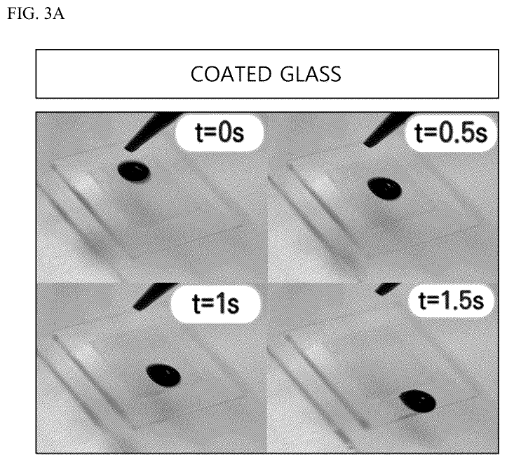

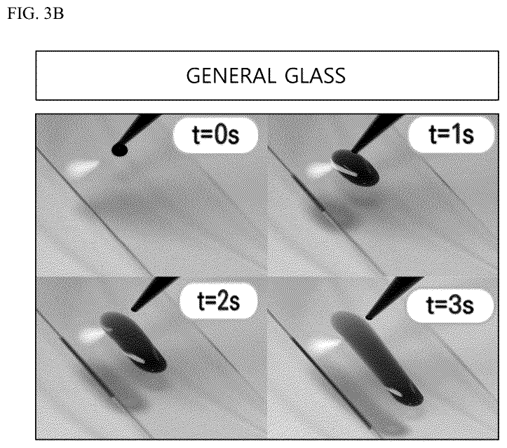

In order to confirm a blood adhesion prevention effect of the coating composition according to the present disclosure, the glass plate to which the coating layer prepared in Example 2 was applied and the uncoated glass plate of Comparative Example 1 were each inclined, and blood was dropped on the surface using a dropper, and the surface conditions of the glass plates were observed over time, which were shown in FIGS. 3A and 3B, respectively.

As shown in FIG. 3A, in the case of the glass plate coated with the coating composition of Example 2 of the present disclosure, it was confirmed that blood stains did not adhere but slid down along the slope to prevent the adhesion of biological materials. On the other hand, as shown in FIG. 3B, in the case of the glass plate of Comparative Example 1, it was confirmed that blood stains adhered.

Therefore, it was confirmed that when the coating composition of the present disclosure was applied to the distal part of the endoscope, the coating composition had a blood stain prevention effect, thereby providing a visibility function of the endoscope.

### Experimental Example 4. Confirmation of Maintenance Ability Under Seawater Conditions

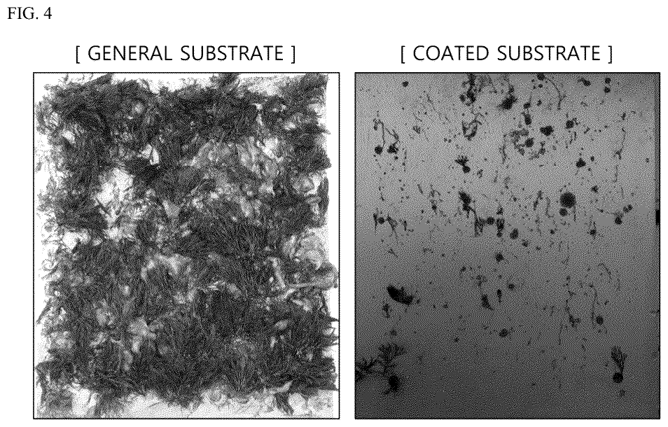

The glass plate to which the coating layer prepared in Example 1 was applied and the uncoated glass plate of Comparative Example 1 were immersed for 45 days at a depth of 3 m below the sea surface at the South Sea Research Institute of the Korea Institute of Ocean Science and Technology, and then raised above the sea surface to observe whether marine organisms adhered to the surface of the glass plate, and the results were shown in FIG. 4.

As shown in FIG. 4, it was confirmed that in the case of a substrate coated with the coating composition of the present disclosure, the adhesion degree of marine organisms was almost not shown, even though the substrate was immersed for 45 days under seawater conditions. In other words, it was confirmed that the coating composition prepared according to Example of the present disclosure was applied to have the ability to maintain the adhesion prevention effect of the marine organisms for a long time even under seawater conditions.

### Experimental Example 5. Confirmation of Endoscope Visibility Effect

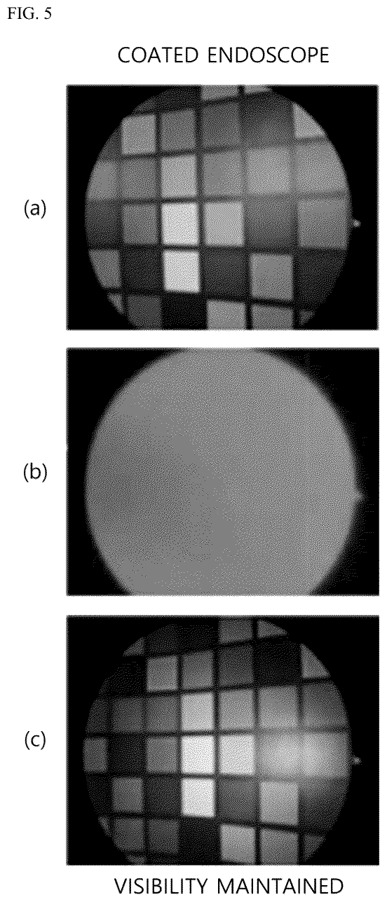

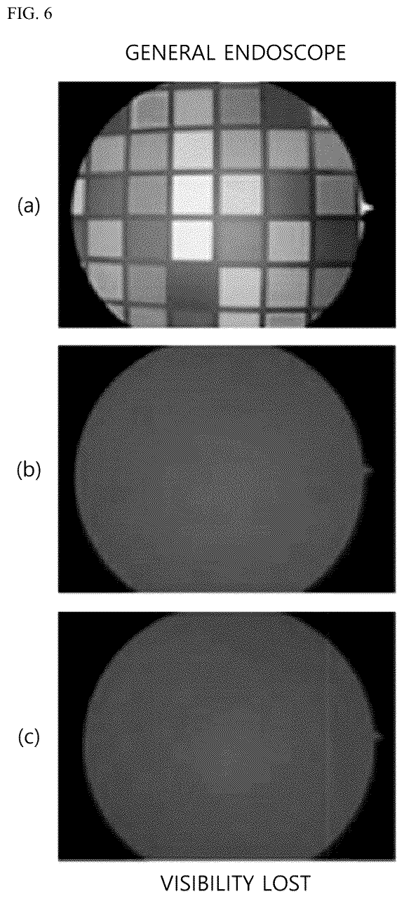

A blood-immersion experiment was conducted using an endoscope using the coated endoscope cap prepared in Example 2 and an endoscope using the uncoated endoscope cap of Comparative Example 2, and the results were shown in FIGS. 5 and 6, respectively.

Specifically, FIGS. 5 and 6 are photographs showing a process of immersing and removing an endoscope in a blood-containing container in sequence. A of FIG. 5 and A of FIG. 6 show the visibility of the endoscope before being immersed in blood, respectively, B of FIG. 5 and B of FIG. 6 show the visibility of the endoscope immersed in blood, respectively, and c of FIG. 5 and C of FIG. 6 show the visibility of the endoscope after the endoscope which had been immersed in the blood was removed from the blood, respectively.

As shown in C of FIG. 5, in the case of the endoscope to which the coating composition according to the present disclosure was applied, it was confirmed that the visibility and maintenance effects were very excellent even after being immersed in blood. On the other hand, as shown in C of FIG. 6, in the case of the general endoscope to which the coating composition was not applied, it was confirmed that the blood was attached and the visibility was lost.

Therefore, it was expected that when the coating composition of the present disclosure was coated on the endoscope, the coating composition would have the effect of solving the problem of not securing the visibility of a medical endoscope due to blood and fogging during surgery.

What is claimed is:

1. A coating composition for visibility of an endoscope comprising:
a stock solution and a diluted solution,
wherein the stock solution includes
an aliphatic hydrocarbon solvent;
a siloxane compound;
an alcohol;
one or more amino silanes; and
a silane-based oligomer.

2. The coating composition for visibility of the endoscope of claim 1, wherein the diluted solution includes an organosilicon compound.

3. The coating composition for visibility of the endoscope of claim 1, wherein a weight mixing ratio of the stock solution and the diluted solution is 1:45 to 55.

4. The coating composition for visibility of the endoscope of claim 1, wherein the coating composition is coated on a distal part of the endoscope.

5. A film for visibility of an endoscope comprising:
a base film; and
the coating composition of claim 1 coated on at least one surface of the base film.

6. The film for visibility of the endoscope of claim 5, wherein a silane-based lubricant is further applied to the at least one surface of the base film.

7. A method for improving visibility of an endoscope, comprising applying the coating composition of claim 1 to a surface of the endoscope.
